(19) United States
(12) Patent Application Publication
(10) Pub. No.: US 2026/0131004 A1
(43) Pub. Date: May 14, 2026

(54) METHOD OF TREATMENT OR INHIBITION

(71) Applicant: Queensland Eye Institute Foundation, Woolloongabba (AU)

(72) Inventors: Traian Vasile Chirila, Robertson (AU); Alexandra Irina Manta, Bulimba (AU); Mark Hughes Burnard Radford, Brisbane (AU); Shuko Suzuki, Ormeau (AU)

(21) Appl. No.: 19/389,820

(22) Filed: Nov. 14, 2025

Related U.S. Application Data
(60) Provisional application No. 63/720,551, filed on Nov. 14, 2024.

(51) Int. Cl. A61K 41/00; A61P 43/00

(52) U.S. Cl. CPC A61K 41/008; A61P 43/00

(57) ABSTRACT

Disclosed are methods of treating eyelid laxity. More particularly, this disclosure relates to a non-invasive method of treating or inhibiting the development or progression of eyelid laxity, comprising exposure of a palpebral conjunctiva of an eye followed by transconjunctival irradiation in the presence of a photosensitizer to Initiate crosslinking in tarsal plate tissue underlying the palpebral conjunctiva.

This application claims priority to U.S. Provisional Patent Application No. 63/720,551 entitled “Method of treatment or inhibition” filed 14 Nov. 2024, the contents of which are incorporated herein by reference in their entirety.

### FIELD OF THE INVENTION

The present invention relates generally to a method of treatment of eyelid laxity. More particularly, this invention relates to a non-invasive method of treatment or inhibition of the development or progression of eyelid laxity comprising exposure of a palpebral conjunctiva of an eye followed by transconjunctival irradiation in the presence of a photosensitizer to initiate crosslinking in tarsal plate tissue underlying the palpebral conjunctiva.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Eyelid laxity, also known as floppy eyelid syndrome, is regarded as a subset of the conditions generated by the laxity of the eyelid [Fowler A M, Dutton J J “Floppy eyelid syndrome as a subset of lax eyelid conditions: Relationships and clinical relevance (an ASOPRS thesis)”, Ophthal. Plast. Reconstr. Surg. 2010; 26; 195-204]. Eyelid laxity can be defined as an acquired hyperelasticity disorder where the inherent rigidity of the lids is lost and can affect both upper and lower eyelids. Due to corneal complications and associations with other eyelid disorders, a floppy eyelid can lead to blindness. Conditions involving eyelid laxity include, for example, involutional ptosis, ectropion and entropion, paralytic ectropion, mechanical distension due to eyelid tumors, atopic disease of the eyelids, blepharochalasis, facial dysmorphism syndromes and hypermobility syndromes. Eyelid laxity is commonly associated with other conditions including obesity, cardiovascular disease, obstructive sleep apnea and eye rubbing.

The main clinical feature of a floppy eyelid is the extreme laxity of the upper or lower tarsus (or tarsal plate), which becomes pliant and rubbery. In normal conditions, the upper or lower tarsal plate is a specialized dense fibrous tissue that is able to display significant rigidity and provide structural integrity, shape and firmness to the entire eyelid. Tarsal plate tissue can be regarded as a unique tissue with transient character possessing features common to both cartilage and fibers [see a description of fibrocartilage tissues in Benjamin M, Ralphs J R “Biology of fibrocartilage cells”, Int. Rev. Cytol. 2004; 233; 1-45]. The tarsal tissue consists mainly of fibrillar collagens type I, III and, to a lesser extent, type VI, as well as elastic networks of fibrillin and elastin fibers [Milz S, Neufang J, Higashiyama I, Putz R, Benjamin M “An immunohistochemical study of the extracellular matrix of the tarsal plate in the upper lid in human beings”, J. Anat. 2005; 206; 37-45; Ezra D G, Beaconsfield M, Collin R “Surgical anatomy of the upper eyelid: old controversies, new concepts”, Expert Rev. Ophthalmol. 2009; 4; 47-57]. In the tarsal plates of the lax eyelids, the collagen expression, structure and distribution remains the same as in normal tarsus [Goldberg R, Seiff S, McFarland J, Simons K, Shorr N “Floppy eyelid syndrome and blefarochalasis”, Am. J. Ophthalmol. 1986; 102; 376-381; and Arrocker-Mettinger E, Haddad R, Konrad K, Steinkogler F J “Floppy eyelid syndrome: Investigations by light and electron microscopies” (Ger.) Klin. Mbl. Augenheilk. 1986; 188; 596-598]. However, the decrease in the amount of elastin and mature elastic fibers observed histopathologically in the tarsal plate of a lax eyelid [Netland P A, Sugrue S P, Albert D M, Shore J W “Histopathologic features of the floppy eyelid syndrome. Involvement of tarsal elastin”, Ophthalmology 1994; 101; 174-181; Schlötzer-Schrehardt U, Stojkovic M, Hofmann-Rummelt C, Cursiefen C, Kruse F E, Holbach L M “The pathogenesis of floppy eyelid syndrome. Involvement of matrix metalloproteinases in elastic fiber degradation”, Ophthalmology 2005; 112; 694-704] is not consistent with an enhanced elasticity and reduced mechanical strength that have been evidenced clinically. A further study [Ezra D G, Ellis J S, Gaughan C, Beaconsfield M, Collin R, Bunce C, Bailly M, Luthert P “Changes in tarsal plate fibrillar collagens and elastic fiber phenotype in floppy eyelid syndrome”, Clin. Exp. Ophthalmol. 2011; 39; 564-571] has demonstrated that instead of a reduction of elastic fibers in the tarsal plates belonging to lax eyelids, the main process consists rather of a change in the phenotype of these fibers. The same study also confirmed that the expression and periodicity of the main fibrillar collagens remain normal.

The current treatments for eyelid laxity include nocturnal lid shielding, taping or lubrication, which generally only provide temporary benefit. Surgical methods, such as full-thickness wedge excision, lateral tarsal strip, lateral canthal tendon plication or lateral tarsorrhaphy, may be used to tighten the lid [Ezra D G, Beaconsfield M, Collin R “Floppy eyelid syndrome: Stretching the limits”, Surv. Ophthalmol. 2010; 55; 35-46]. However, these surgical horizontal tightening methods usually only provide temporary benefit as the eyelid tissue continues to stretch with time and the eyelid laxity returns. Furthermore, these surgical procedures are usually performed under general anesthesia. This is a particular problem as many patients with floppy eye syndrome are commonly also afflicted by cardiovascular disease, obesity and sleep apnea, which require particular considerations with respect to administration of general anesthesia.

Australian Patent No. 2018201200 discusses techniques for treating eyelid laxity involving exposure of a tarsal plate, followed by application of a photosensitizer and irradiation. This method requires surgical exposure of the tarsal plate prior to photosensitizer application.

There is a need for non-invasive methods for treating or inhibiting the development or progression of eyelid laxity.

### SUMMARY OF THE INVENTION

This invention is predicated in part on the discovery that exposure of a palpebral conjunctiva (also known as the tarsal conjunctiva), which is the mucous membrane lining the inside of an eyelid, to radiation in the presence of a photosensitizer initiates crosslinking within the underlying tarsal plate, and particularly crosslinking of collagen in the tarsal plate tissue. This crosslinking has been found to improve or restore strength or rigidity to the tarsal plate resulting in arresting or slowing the progression of eyelid laxity. Accordingly, the inventors have conceived that non-invasive exposure of a palpebral conjunctiva, for example, by everting an eyelid, application of a photosensitizer that initiates crosslinking in response to photo-activating radiation to the exposed palpebral conjunctiva, followed by irradiation with photo-activating radiation may be used to treat or inhibit the progression or development of eyelid laxity. This method provides a non-invasive protocol for the treatment or inhibition of the progression or development of eyelid laxity.

Accordingly, in one aspect, there is provided a method of treating or inhibiting the development or progression of eyelid laxity in a subject comprising:
a) exposing a palpebral conjunctiva of an eye;
b) applying to at least part of the exposed palpebral conjunctiva a photosensitizer that initiates crosslinking in response to photo-activating radiation; and
c) irradiating the exposed palpebral conjunctiva with photo-activating radiation to initiate crosslinking in tarsal plate tissue underlying the palpebral conjunctiva.

In some embodiments, the photosensitizer is in solubilized form. In some embodiments, the photosensitizer is selected from the group consisting of riboflavin, acridine orange, Quantacure QTX, protoporphyrin IX and pharmaceutically acceptable salts, derivatives and solvates thereof. In some embodiments, the photosensitizer absorbs radiation at a wavelength in the range of from about 300 to about 500 nm. In particular embodiments, the photosensitizer is riboflavin or a pharmaceutically acceptable salt, derivative or solvate thereof; including riboflavin 5′-phosphate or a pharmaceutically acceptable salt or solvate thereof.

In some embodiments, the photo-activating radiation is radiation with a wavelength in the range of from about 300 to about 500 nm. In some embodiments, the photo-activating radiation is UV-A radiation, such as radiation with a wavelength of about 365 nm.

In some embodiments, the palpebral conjunctiva is exposed by everting an upper or lower eyelid of the subject to expose the palpebral conjunctiva, such as by using a lid retractor.

In particular embodiments, the method further includes delivery of O₂ gas at a site of irradiation.

Also provided, in another aspect, is a use of a photosensitizer that initiates crosslinking in response to photo-activating radiation in the manufacture of a medicament for treating or inhibiting the development or progression of eyelid laxity in a subject, wherein the photosensitizer is to be applied to at least part of an exposed palpebral conjunctiva of an eye of the subject, and the exposed palpebral conjunctiva is to be irradiated with photo-activating radiation to initiate crosslinking in tarsal plate tissue underlying the palpebral conjunctiva.

In a further aspect, there is provided a use of a photosensitizer that initiates crosslinking in response to photo-activating radiation for treating or inhibiting the development or progression of eyelid laxity in a subject, wherein the photosensitizer is to be applied to at least part of an exposed palpebral conjunctiva of an eye of the subject, and the exposed palpebral conjunctiva is to be irradiated with photo-activating radiation to initiate crosslinking in tarsal plate tissue underlying the palpebral conjunctiva.

In yet another aspect, there is provided a photosensitizer that initiates crosslinking in response to photo-activating radiation for use in treating or inhibiting the development or progression of eyelid laxity in a subject, wherein the photosensitizer is applied to at least part of an exposed palpebral conjunctiva of an eye of the subject, and the exposed palpebral conjunctiva is irradiated with photo-activating radiation to initiate crosslinking in tarsal plate tissue underlying the palpebral conjunctiva.

### BRIEF DESCRIPTION OF THE DRAWINGS

FIG. 1 is a diagram outlining the method of the invention. Radiation and the photosensitizer penetrate across the palpebral conjunctiva (p.c.) to reach the tarsus.

FIG. 2 is a series of histologic photomicrographs of human tarsus samples, using 3 different staining procedures, in nonirradiated samples (FIGS. 2A, C, E and G) and in samples irradiated with ultraviolet-A light (wavelength 365 nm, irradiance 75 mW/cm², and fluence 13.5 J/cm²) (FIGS. 2B, D, F and H). Magnification in all photomicrographs: x100. Photomicrographs showing normal appearance of meibomian glands, meibocytes, and surrounding tissue (H&E stain) in nonirradiated samples #16 (FIG. 2A) and #18 (FIG. 2E), and in irradiated samples #17 (FIG. 2B) and #19 (FIG. 2F). Photomicrographs showing normal appearance of collagen network (red) and Meibomian glands (Van Gieson stain) in nonirradiated sample #16 (FIG. 2C) and in irradiated sample #17 (FIG. 2D). Masson trichrome staining showing normal appearance of collagen network (blue) and meibomian glands in nonirradiated sample #18 (FIG. 2G) and in irradiated sample #19 (FIG. 2H).

FIG. 3 is a series of histologic photomicrographs of human tarsal conjunctiva specimens stained with the H&E protocol: nonirradiated sample #18 (FIG. 3A); irradiated sample #17 (FIG. 3B); and irradiated sample #19 (FIG. 3C). The samples were irradiated with ultraviolet-A light (wavelength 365 nm, irradiance 75 mW/cm², and fluence 13.5 J/cm²). Magnification in all photomicrographs: x200.

FIG. 4 is a series of images of sheep eyelid tissue permeated with the photosensitizer and exposed to UV-A radiation. FIG. 4A is a photographic image of an eyelid tissue sample permeated with photosensitizer solution. FIG. 4B to 4D are thermographic images recorded before (FIG. 4B) and after exposure to UV-A radiation (365 nm, 250 mW/cm²) for 60 s (FIG. 4C) and 180 s (FIG. 4D).

FIG. 5 is a series of graphs showing the temperature on the conjunctival surface of sheep eyelid tissue. FIG. 5A is a plot of the temperature measured on the conjunctival surface as a function of exposure time at different irradiances following photosensitizer treatment. FIG. 5B is a plot of the maximum temperature increase (ΔT) recorded on conjunctival surface as a function of fluence following photosensitizer treatment. ΔT is defined as the difference between the temperature attained at the end of exposure time, i.e. after 3 min, and the baseline temperature recorded prior to commencing irradiation.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogues and the like. When the above term is used, then it is to be understood that this includes the active agent per se as well as pharmaceutically acceptable, pharmacologically active salts, esters, amides, prodrugs, metabolites, analogues, etc.

As used herein, the term “and/or” refers to and encompasses any and all possible combinations of one or more of the associated listed items, as well as the lack of combinations when interpreted in the alternative (or).

The phrase “aqueous carrier” is used herein to refer to a liquid aqueous diluent, wherein the aqueous carrier includes, but is not limited to, water, saline, aqueous buffer and aqueous solutions comprising water soluble or water miscible additives such as glucose or glycerol. The aqueous carrier may also be in the form of an oil-in-water emulsion.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified for the listed elements. Thus, the phrase “consisting essentially of” indicates that the listed elements are required or mandatory, but that other elements are optional and may or may not be present depending upon whether or not they affect the activity or action of the listed elements.

As used herein, the terms “condition” or “disease” refers to an abnormality in the physical state of the body as a whole or one of its parts.

When used herein, the term “crosslinker” or “crosslinking agent” refers to a chemical moiety that can chemically join two or more molecules, for example by covalent bonding or ionic bonding, preferably by covalent bonding. An example of a crosslinking agent is O₂ which acts as a crosslinking agent when in the form of its high energy singlet state. A crosslinker or crosslinking agent will preferably be pharmaceutically acceptable.

By “derivative” is meant a molecule, such as a small molecule, that has been derived from the basic molecule by modification, for example by conjugation or complexing with other chemical moieties as would be understood in the art. The term “derivative” also includes within its scope alterations that have been made to a parent molecule including additions or deletions that provide for functionally equivalent molecules.

By “effective amount”, in the context of treating eyelid laxity is meant the application of an amount of an agent or composition to an individual in need of such treatment, that is effective for preventing incurring a symptom, holding in check such symptom(s) and/or treating existing symptom(s) of eyelid laxity. In the context of inhibiting the development or progression of eyelid laxity, an “effective amount” means an amount of an agent or composition that is effective for preventing incurring a symptom or holding in check such symptom(s). The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The term “eyelid laxity” or “floppy eyelid syndrome” when used herein refers to conditions where an acquired hyperelasticity disorder in an eyelid has resulted in the inherent rigidity of the eyelid being lost.

The term “inhibit the development or progression of” refers to a treatment which increases the resistance of a subject to developing the disease, disorder or condition or, in other words, decreases the likelihood that the subject will develop the disease, disorder or condition, as well as a treatment after the disease, disorder or condition has begun in order to reduce or eliminate it altogether or prevent it from becoming worse. This phrase also includes within its scope preventing the disease, disorder or condition from occurring in a subject which may be predisposed to the disease, disorder or condition but has not yet been diagnosed as having it.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction.

Similarly, a “pharmacologically acceptable” salt, ester, amide, prodrug, compound or derivative of a compound as provided herein is a salt, ester, amide, prodrug, compound or derivative that this not biologically or otherwise undesirable.

The term “photo-activating radiation” when used herein refers to radiation that can activate a photosensitizer to produce a chemical change in another molecule. Suitably the photosensitizer absorbs radiation at a wavelength of the photo-activating radiation. In some embodiments the radiation is radiation at a wavelength in the range of from about 300 to about 500 nm, especially UV-A radiation.

When used herein, the term “photosensitizer” refers to a molecule that, on irradiation by photo-activating radiation, produces a chemical change in another molecule through a photochemical process. Examples of “another molecule” include, for example, a crosslinker or crosslinking agent such as O₂. A photosensitizer may convert O₂ molecules from the normal O₂ triplet state to a more energetic singlet state that can initiate crosslinking, for example in tissue molecules or macromolecules. Further examples of “another molecule” include tissue molecules or macromolecules, including collagen macromolecules. A photosensitizer, after exposure to radiation and transition to a more energetic state, may also produce a chemical change in collagen and/or other tissue molecules and initiate or generate crosslinking in the tissue. The skilled person will appreciate that optimum results will be achieved when the selected photosensitizer absorbs radiation at a wavelength of the photo-activating radiation. The absorption wavelength(s) of a photosensitizer can be determined by ultraviolet/visible (UV/VIS) spectrophotometry using a commercially available UV/VIS spectrophotometer in accordance with well known procedures. A photosensitizer will preferably be pharmaceutically acceptable, non-irritant and non-toxic.

As used herein, the term “salts”, “derivative” and “solvate” include any pharmaceutically acceptable salt, derivative, or solvate or any other compound which, upon administration to the recipient, is capable of providing (directly or indirectly) a desired photosensitizer. Suitable pharmaceutically acceptable derivatives include esters, such as phosphate esters. Suitable pharmaceutically acceptable salts include salts of pharmaceutically acceptable inorganic acids such as hydrochloric, sulfuric, phosphoric, nitric, carbonic, boric, sulfamic and hydrobromic acids, or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium, particularly sodium. Also, basic nitrogen-containing groups may be quaternized with such agents as lower alkyl halides, such as methyl, ethyl, propyl and butyl chlorides, bromides and iodides; dialkyl sulfates such as dimethyl and diethyl sulfate; and others. Pharmacologically acceptable solvates are known in the art, and include hydrates and alcoholates. Suitably, pharmaceutically acceptable solvates include hydrates, for example monohydrates, dihydrates and trihydrates. The skilled person will understand that a photosensitizer may be in the form of a pharmaceutically acceptable salt, and/or a solvate and/or a derivative, for example riboflavin 5′-phosphate monosodium salt dihydrate and the like. The preparation of salts, derivatives and solvates can be carried out by methods well known in the art. Lists of suitable salts are found in, for example, Allen L V (Ed.) “Remington: The Science and Practice of Pharmacy”. The Pharmaceutical Press, London, 2012, 22^{nd} edition; Stahl P H and Wermuth C G (Eds.) “Pharmaceutical Salts: Properties, Selection, and Use”. Wiley-VCH, Germany, 2002, 2^{nd} edition; and Berge S M et al. “Pharmaceutical salts”. J. Pharm. Sci. 1977; 66:1-19, each of which is incorporated herein by reference in its entirety.

The term “simultaneously” denotes that the two agents are applied at substantially the same time.

As used herein, the phrase “solubilized form” refers to a form where a compound, such as a photosensitizer, is dissolved in a liquid such that a solution comprising a uniform distribution of the compound is obtained which is substantially free of solid compound. In some embodiments, the liquid is an aqueous carrier as described herein.

The term “subject” or “individual” as used herein refers to a vertebrate subject, particularly a mammalian or avian subject, for whom therapy or prophylaxis is desired. Suitable subjects include, but are not limited to, primates; avians (birds); livestock animals such as sheep, cows, horses, deer, donkeys and pigs; laboratory test animals such as rabbits, mice, rats, guinea pigs and hamsters; companion animals such as cats and dogs; and captive wild animals such as foxes, deer and dingoes. In particular embodiments, the subject is a primate, suitably a human. However, it will be understood that the aforementioned terms do not imply that symptoms are present.

As used herein, the terms “treatment”, “treating”, and the like, refer to obtaining a desired pharmacologic and/or physiologic effect. The effect may be therapeutic in terms of a partial or complete cure for a disease, disorder or condition and/or adverse effect attributable to the disease, disorder or condition. These terms also cover any treatment of a condition or disease in a subject, particularly in a human, and include: (a) inhibiting the disease, disorder or condition, i.e. arresting its development; (b) relieving the disease, disorder or condition, i.e. causing regression of the disease, disorder or condition; or (c) relieving one or more symptoms of the disease, disorder or condition.

As used herein “water soluble form” refers to a chemical and/or physical form of a compound, such as a photosensitizer, where the compound or a salt, derivative, solvate and/or polymorph thereof has sufficient solubility in water at ambient temperature to achieve a concentration of from about 0.01 to about 20% w/v. Solubility can be determined using methods well known in the art.

Each embodiment described herein is to be applied mutatis mutandis to each and every embodiment unless specifically stated otherwise.

### 2. Methods of Treatment

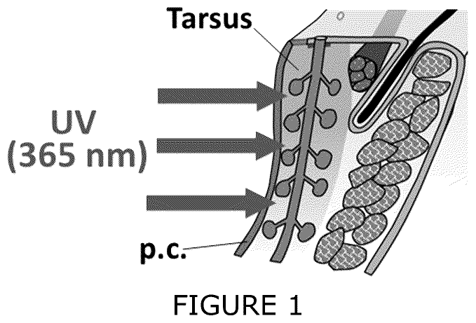

The present invention is based, in part, on the identification that exposure of a palpebral conjunctiva to radiation in the presence of a photosensitizer initiates crosslinking within the underlying tarsal plate, and particularly crosslinking of collagen in the tarsal plate tissue. This crosslinking has been found to improve or restore strength or rigidity to the tarsal plate resulting in arresting or slowing the progression of eyelid laxity. Thus, the inventors conceived that a non-invasive treatment of eyelid laxity may be achieved by exposing a palpebral conjunctiva and irradiating the palpebral conjunctiva with photo-activating radiation in the presence of a photosensitizer. A diagram demonstrating the application of the radiation to the palpebral conjunctiva is provided in FIG. 1.

Accordingly, the present invention provides a method of treating or inhibiting the development or progression of eyelid laxity in a subject comprising, consisting or consisting essentially of:
a) exposing a palpebral conjunctiva of an eye;
b) applying to at least part of the exposed palpebral conjunctiva a photosensitizer that initiates crosslinking in response to photo-activating radiation; and
c) irradiating the exposed palpebral conjunctiva with photo-activating radiation to initiate crosslinking in tarsal plate tissue underlying the palpebral conjunctiva.

The methods of the invention may be applied to the palpebral conjunctiva of an upper eyelid, a lower eyelid or both eyelids, subsequently or simultaneously. The methods may be applied to one or both eyes of a subject, individually, subsequently or simultaneously. In particular embodiments, the methods are applied to both eyes of a subject.

A person of skill in the art will be well aware of suitable methods for exposing a palpebral conjunctiva of an eye of the subject. For example, the palpebral conjunctiva may be exposed by everting an eyelid of the subject, such as an upper and/or lower eyelid of the subject. Suitable techniques include, but are not limited to, a device such as a lid retractor, or an item pressed on the eyelid at the upper edge of the tarsal plate for the superior eyelid or the lower edge for the inferior eyelid, such as a cotton bud, a finger or a glass rod with manual eversion of the eyelid to expose the palpebral conjunctiva. Suitable techniques are discussed, for example, in Wolffsohn J S, Tahhan M, Vidal-Rohr, et al. “Best technique for upper lid eversion”. Contact Lens and Anterior Eye. 2019; 42:666-669. In some embodiments, the palpebral conjunctiva is exposed using a lid retractor.

An exemplary procedure for everting an eyelid of a subject includes:
a) instructing the subject to look down without closing the eyes;
b) pulling the eyelid slightly downward and away from the globe of the eye to create tension;
c) if using a finger or cotton-tipped applicator, placing the finger or cotton-tipped applicator at the upper edge of the tarsal plate through the eyelid, approximately 10 mm above the eyelid margin on the upper eyelid or 4 mm below the eyelid margin on the lower eyelid; and
d) pressing gently on the tarsal plate edge through the eyelid while lifting the eyelid margin upward and over the pressure point to expose the palpebral conjunctiva overlying the entire tarsal plate.

The photosensitizer is suitably pharmaceutically acceptable, substantially non-toxic and substantially non-irritant. In particular embodiments, the photosensitizer is water soluble. The skilled person will readily understand that different photosensitizers will absorb photosensitizing radiation of specific wavelengths according to the chemical structure of the chromophore, and will be able to match the photosensitizer to the appropriate wavelength of photosensitizing radiation. In some embodiments, the photosensitizer absorbs radiation in the ultraviolet region of the electromagnetic spectrum, especially radiation at a wavelength in the range of from about 300 to about 500 nm (and all integers therebetween) or a wavelength in the range of from about 300 to about 400 nm. In preferred embodiments, the photosensitizer absorbs long wavelength ultraviolet radiation e.g. UV-A radiation (about 320 to about 400 nm). In particular embodiments, the photosensitizer absorbs radiation at a wavelength of about 320, 325, 330, 335, 340, 345, 350, 355, 360, 365, 370, 375, 380, 385, 390, 395 or 400 nm; especially about 365 nm. In some embodiments, the photosensitizer absorbs radiation at a wavelength of about 450 to about 495 nm (blue light). In other embodiments, the photosensitizer absorbs radiation at a wavelength of about 495 to about 570 nm (green light). In preferred embodiments, the photosensitizer has regulatory approval for food and/or drug use. While the use of more than one photosensitizer is encompassed herein, in particular embodiments, there is only a single photosensitizer molecule present.

In some embodiments, the photosensitizer comprises riboflavin or a pharmaceutically acceptable salt, derivative or solvate thereof. Riboflavin is also known as vitamin B₂, and has the IUPAC name 7,8-dimethyl-10-[(2S,3S,4R)-2,3,4,5-tetrahydroxypentyl] benzo[g]pteridine-2,4-dione. While any form of riboflavin is suitable, in preferred embodiments, riboflavin is in a water soluble form, for example as a water soluble derivative, salt or solvate, such as an alkali metal salt of riboflavin 5′-phosphate. In particular embodiments, the photosensitizer comprises a sodium salt of riboflavin 5′-phosphate or a pharmaceutically acceptable solvate thereof, such as riboflavin 5′-phosphate monosodium salt. When in the form of a solvate, the solvate is preferably a hydrate.

In some embodiments, the photosensitizer is rose bengal (4,5,6,7-tetrachloro-2′,4′,5′,7′-tetraiodofluorescein disodium salt). Preferably rose bengal is used in conjunction with irradiation by green light, i.e. a radiation wavelength of about 495 to about 570 nm.

In some embodiments, the photosensitizer is selected from the group consisting of lucigenin, acridine orange, riboflavin, Quantacure QTX (2-hydroxy-3-(3,4-dimethyl-9-oxo-9H-thioxanthen-2-yloxy)-N, N, N-trimethyl-1-propanaminium chloride), erythrosine B, protoporphyrin IX and pharmaceutically acceptable salts, derivatives and solvates thereof; especially riboflavin, acridine orange, Quantacure QTX, protoporphyrin IX or pharmaceutically acceptable salts, derivatives and solvates thereof. In such embodiments, the irradiation may have a wavelength of greater than about 300 nm, such as from about 300 nm to about 500 nm (e.g. UV-A or blue light).

In some embodiments, the photosensitizer is selected from the group consisting of Lissamine green B, Brilliant blue G, trypan blue, rose bengal and pharmaceutically acceptable salts, derivatives and solvates thereof. In such embodiments, the irradiation may have a wavelength of greater than about 400 nm, such as from about 495 nm to about 570 nm (e.g. green light).

In some embodiments, the photosensitizer is selected from the group consisting of riboflavin, rose bengal, lucigenin, acridine orange, Quantacure QTX, Lissamine green B, Brilliant blue G, trypan blue, erythrosine B, protoporphyrin IX and pharmaceutically acceptable salts, derivatives and solvates thereof; especially riboflavin, acridine orange, Quantacure QTX, protoporphyrin IX, lucigenin, rose bengal, erythrosine B and pharmaceutically acceptable salts, derivatives and solvates thereof; more especially riboflavin, acridine orange, Quantacure QTX, protoporphyrin IX and pharmaceutically acceptable salts, derivatives and solvates thereof. In preferred embodiments, the photosensitizer is riboflavin or a pharmaceutically acceptable salt, derivative or solvate thereof, such as riboflavin 5′-phosphate or a pharmaceutically acceptable salt or solvate thereof.

In some embodiments, the photosensitizer is applied to at least part of the exposed palpebral conjunctiva. Suitably the photosensitizer is applied to at least 25%, at least 50%, at least 75%, at least 80% or at least 90% of the surface of the exposed palpebral conjunctiva. Preferably the photosensitizer is applied to substantially the entire surface of the exposed palpebral conjunctiva.

In some embodiments, the photosensitizer is in solubilized form. Preferably, the photosensitizer is water soluble.

While the photosensitizer may be applied in a number of suitable forms, in particular embodiments, the photosensitizer is formulated in a carrier, such as a liquid or gel carrier. In such embodiments, the photosensitizer may be administered in a pharmaceutical composition comprising the photosensitizer and a pharmaceutically acceptable carrier.

The photosensitizer is preferably in a form suitable for topical application to the palpebral conjunctiva, such as an emulsion, solution or gel; especially a solution. Techniques for formulation and administration may be found in, for example, Allen L V (Ed.) “Remington: The Science and Practice of Pharmacy”. The Pharmaceutical Press, London, 2012, 22^{nd} edition.

In some embodiments, the photosensitizer is formulated as an aqueous composition, for example an aqueous solution, an aqueous gel, or an oil in water emulsion, preferably as an aqueous solution. In particular embodiments, the photosensitizer is present in the composition in an amount of about 0.01 to about 20% w/v (and all integers therebetween), such as about 0.1% to 10% w/v, about 0.1% to 5% w/v, about 0.1% to 2% w/v, about 0.1% to 1% w/v, about 0.1% to 0.5% w/v or about 0.1% to 0.3% w/v. In some embodiments, the photosensitizer is present in an amount of about 0.1%, 0.2% or 0.3 w/v.

In some embodiments, the carrier is an aqueous carrier. The aqueous carrier is preferably a pharmaceutically acceptable aqueous carrier. A variety of pharmaceutically acceptable aqueous carriers well known in the art may be used. Exemplary aqueous carriers include, but are not limited to, saline, water, aqueous buffer, an aqueous solution comprising water and a miscible solvent, and combinations thereof. In particular embodiments, the aqueous carrier is saline. When saline is used, it is preferably isotonic for the point of administration. For example, in some embodiments the saline comprises 0.15 to 8% w/v sodium chloride (and all one hundredth integer percentages therebetween); especially 0.18% to 7% w/v, 0.22% to 5% w/v or 0.45% to 3% w/v sodium chloride; more especially 0.5 to 2% w/v or 0.65% to 1.5% w/v sodium chloride; most especially about 0.9% w/v sodium chloride.

In some embodiments where the aqueous carrier is not isotonic, for example water, the composition may contain a tonicity agent. Any pharmaceutically acceptable tonicity agent well known in the art may be used. Suitable tonicity agents include, but are not limited to, boric acid, sodium acid phosphate buffer, sodium chloride, glucose, trehalose, potassium chloride, calcium chloride, magnesium chloride, poly(propylene glycol), glycerol, mannitol, or salts or combinations thereof. The tonicity agent may be present in the composition in an amount that provides isotonicity with the point of administration, for example in the range of from 0.02 to 15% w/v (and all one hundredth integer percentages therebetween).

In some embodiments the carrier is a buffer, wherein the buffer maintains a pH in the range of from 6 to 8, 6.5 to 7.5 or about 7. Suitable buffering agents include, but are not limited to, acetic acid, citric acid, sodium metabisulfite, histidine, sodium bicarbonate, sodium hydroxide, boric acid, borax, alkali metal phosphates, phosphate or citrate buffers, or combinations thereof. The buffering agent may be present in the composition in an amount suitable to maintain the desired pH.

The composition may further comprise a rheology modifier. The rheology modifier may be used to alter the surface tension and flow of the composition. Suitable rheology modifiers are well known in the art. For example, the rheology modifier may be selected from, but is not limited to, hyaluronic acid, chitosan, poly(vinyl alcohol), poly(ethylene glycol), poly(vinyl pyrrolidone), dextran, methylcellulose, hydroxypropylmethylcellulose, hydroxyethylcellulose, hydroxypropylcellulose, hydroxypropyl guar, acrylates such as Carbopol polymers, poloxamers, gum arabic, xanthan gum, guar gum, locust bean gum, carboxymethylcellulose, alginate, starch (from rice, corn, potato or wheat), carrageenan, konjac, aloe vera gel, agarose, pectin, tragacanth, curdlan gum, gellan gum, scleroglucan, and derivatives and combinations thereof. The rheology modifier should be present in an amount sufficient to obtain the desired viscosity of the composition. The rheology modifier may be present in an amount in the range of from about 0.5% to 5% w/v of the composition (and all one tenth integer percentages therebetween).

The composition may further comprise a surfactant. A variety of pharmaceutically acceptable surfactants well known in the art may be used. Exemplary surfactants include, but are not limited to, surfactants of the following classes: alcohols; amine oxides; block polymers; carboxylated alcohol or alkylphenol ethoxylates; carboxylic acids/fatty acids; ethoxylated arylphenols; ethoxylated fatty esters, oils, fatty amines or fatty alcohols such as cetyl alcohol; fatty esters; fatty acid methyl ester ethoxylates; glycerol esters such as glycerol monostearate; glycol esters; lanolin-based derivatives; lecithin or derivatives thereof; lignin or derivatives thereof; methyl esters; monoglycerides or derivatives thereof; poly(ethylene glycol) s; poly(propylene glycol) s; alkylphenol poly(ethylene glycol) s; alkyl mercaptan poly(ethylene glycol) s; poly(propylene glycol) ethoxylates; poly(ethylene glycol) ethers such as Cetomacrogol 1000; polymeric surfactants; propoxylated and/or ethoxylated fatty acids, alcohols or alkylphenols; protein-based surfactants; sarcosine derivatives; sorbitan derivatives such as polysorbates; sorbitol esters; esters of sorbitol polyglycol ethers; fatty acid alkylolamides; N-alkylpolyhydroxy fatty acid amide; N-alkoxypolyhydroxy fatty acid amide; alkyl polyglycosides; quaternary ammonium compounds such as benzalkonium chloride; cyclodextrins such as alpha-, beta- or gamma-cyclodextrin; sucrose or glucose esters or derivatives thereof; sulfosuccinates such as dioctyl sodium sulfosuccinate; or combinations thereof.

The composition of the invention may further comprise any other pharmaceutically acceptable excipients commonly present in pharmaceutical formulations. For example, the composition may further comprise an alcohol such as isopropanol, benzyl alcohol, cetearyl alcohol or ethanol; a polysaccharide such as chitosan, chitin, dermatan, hyaluronate, heparin, chondroitin, cyclodextrin or derivatives thereof; or combinations thereof.

Suitable water soluble photosensitizers are readily available from commercial sources, such as Sigma Aldrich, Inc. (Merck KGAA, Darmstadt, Germany), Cayman Chemicals (Ann Arbor, Michigan, USA) and ThermoFisher Scientific (Waltham, MA, USA). For example, riboflavin 5′-phosphate sodium salt, riboflavin 5′-phosphate sodium salt hydrate and riboflavin 5′-phosphate sodium salt dihydrate are available from Sigma Aldrich and Cayman Chemicals. Aqueous compositions, such as solutions, of a photosensitizer, for example a water soluble form of riboflavin, are commercially available or may be prepared using known methods. For example, a water soluble salt, derivative or solvate of riboflavin may be dissolved in an aqueous pharmaceutically acceptable carrier selected from, but not limited to, saline, water, aqueous buffer, an aqueous solution comprising water and a miscible solvent, and combinations thereof. An aqueous composition may additionally include other pharmaceutically acceptable excipients as described above. Photosensitizers formulated as a solution are commercially available, for example ParaCel™ (Avedro, Inc, Waltham, MA, USA) is a commercially available aqueous solution comprising 0.25% riboflavin in the form of riboflavin 5′-phosphate sodium salt with hydroxypropylmethylcellulose and benzalkonium chloride, and VibeX Rapid™ (Avedro, Inc) is a commercially available solution comprising 0.1% riboflavin 5′-phosphate sodium salt, together with saline and hydroxypropylmethylcellulose. Other commercially available riboflavin solutions include VibeX Xtra™ (0.22% riboflavin, saline isotonic); MedioCROSS TE (0.25% riboflavin 5′-phosphate, 1.2% hydroxypropylmethylcellulose, 0.01% benzalkonium chloride); MedioCROSS M (0.1% riboflavin 5′-phosphate, 1.1% hydroxypropylmethylcellulose); Photrexa (0.146% riboflavin 5′-phosphate ophthalmic solution); or Photrexa viscous (0.146% riboflavin 5′-phosphate in 20% dextran ophthalmic solution), all available from Avedro, Inc, Waltham, MA, USA.

In some embodiments the photosensitizer is commercially available as a sterile formulation in a dispenser, for example as a sterile aqueous sodium riboflavin 5′-phosphate solution packaged in a syringe, ampoule, vial or dropper, for example a single use syringe.

The skilled person will understand that the amount of photosensitizer required will depend on the identity of the photosensitizer. In some embodiments the amount of photosensitizer applied to the exposed palpebral conjunctiva is from 1 to 5 ml (and all integers therebetween) of a solution comprising from 0.01 to 0.5% w/v of photosensitizer (and all integers therebetween), suitably 0.1 to 0.25% w/v. In some embodiments the amount of riboflavin applied to an exposed palpebral conjunctiva is 1.8 mL of 0.25% riboflavin (as riboflavin 5′-phosphate) in aqueous carrier; 2 ml of 0.22% riboflavin (as riboflavin 5′-phosphate) in aqueous carrier; 3 mL of 0.146% riboflavin (as riboflavin 5′-phosphate) in aqueous carrier; or 2 mL of 0.1% riboflavin (as riboflavin 5′-phosphate) in aqueous carrier.

The photosensitizer is applied to the exposed palpebral conjunctiva using any suitable means known to the skilled person. In particular embodiments, the photosensitizer is topically applied to the palpebral conjunctiva. In some embodiments, the photosensitizer is applied using an applicator. In some embodiments, the photosensitizer is applied dissolved in an aqueous carrier, preferably as an aqueous solution as discussed herein. In some embodiments, the photosensitizer is instilled directly onto the surface of the exposed palpebral conjunctiva. In some embodiments the photosensitizer is instilled onto the palpebral conjunctiva using, a suitable applicator such as a syringe, pipette or dropper. In alternative embodiments, the photosensitizer solution is first absorbed onto an absorbent material, such as a disposable textile pad, for example a surgical sponge or neurosurgical patty, prior to application to the exposed palpebral conjunctiva. In some embodiments, the photosensitizer is applied to substantially the entire exposed surface of the palpebral conjunctiva.

Optionally, excess photosensitizer may be removed from the palpebral conjunctiva prior to irradiation if desired. For example, excess photosensitizer solution may be removed from the palpebral conjunctiva using absorbent material, such as surgical sponge.

The photosensitizer may be applied to the palpebral conjunctiva simultaneously with or prior to irradiation, especially prior to irradiation. For example, the photosensitizer may be applied from about 30 seconds to about 60 minutes (and all seconds and minutes therebetween) prior to irradiation, such as from about 1 minute to 60 minutes, 2 minutes to 60 minutes, 2 minutes to 30 minutes, 2 minutes to 20 minutes, 2 minutes to 10 minutes, 2 minutes to 5 minutes and the like, including about 2, 3, 4, 5, 6, 7, 8, 9, 10, 15, 20, 25 or 30 minutes prior to irradiation. In some embodiments, the photosensitizer is applied from about 2, 3, 4 or 5 minutes prior to irradiation. Where required, additional photosensitizer may be applied to the palpebral conjunctiva during irradiation.

In some embodiments, the photosensitizer is allowed to remain in contact with the exposed palpebral conjunctiva for a period of from about 30 seconds to about 60 minutes (and all seconds and minutes therebetween), such as from about 1 minute to 60 minutes, 2 minutes to 60 minutes, 2 minutes to 30 minutes, 2 minutes to 20 minutes, 2 minutes to 10 minutes, 2 minutes to 5 minutes and the like, including about 2, 3, 4, 5, 6, 7, 8, 9, 10, 15, 20, 25 or 30 minutes. In some embodiments, the photosensitizer is allowed to remain in contact with the exposed palpebral conjunctiva for a period of from about 2, 3, 4 or 5 minutes. The exact timing will depend on the amount and concentration of the photosensitizer and can be easily determined by the skilled person based on the concentration of the photosensitizer solution. In some embodiments, the photosensitizer applied to the palpebral conjunctiva is 0.1% riboflavin (as riboflavin 5′-phosphate, sodium salt) in aqueous solution which is allowed to contact the palpebral conjunctiva for approximately 2 to 5 minutes, such as 2, 3, 4 or 5 minutes. In some embodiments the photosensitizer applied to the palpebral conjunctiva is 0.25% riboflavin (as riboflavin 5′-phosphate, sodium salt) in aqueous solution which is allowed to contact the palpebral conjunctiva for approximately 2 to 5 minutes, such as 2, 3, 4 or 5 minutes. Additional photosensitizer solution may be applied during this time, for example, to ensure that the exposed palpebral conjunctiva is wet and covered with the solution for the desired duration.

After the photosensitizer has contacted the palpebral conjunctiva for the required length of time, the absorbent material, or excess solution, is optionally removed and the exposed palpebral conjunctiva is exposed to radiation of appropriate wavelength for the photosensitizer used. Suitable wavelengths are discussed supra. In particular embodiments, the photosensitizer is a sodium salt of riboflavin 5′-phosphate and the radiation wavelength is in the range of from about 300 to about 500 nm (and all integers therebetween) or from about 300 and about 400 nm, especially UV-A radiation of a wavelength of about 320 to about 400 nm. In preferred embodiments, the radiation wavelength is about 365 nm.

The skilled person will understand that the irradiation time necessary to induce sufficient crosslinking will be dependent on several factors including the irradiance intensity delivered by the radiation source (mW/cm²), and the beam width. Preferably, use of an irradiance of about 3 mW/cm² up to about 150 mW/cm² is envisaged. It will also be appreciated that the radiant exposure should not be detrimental to the health of the tissue, particularly the palpebral conjunctiva. A radiant exposure, or fluence, of about 4 to about 27 J/cm² is considered appropriate. In some embodiments, the radiant exposure of the palpebral conjunctiva, or fluence, is from about 5 to about 8 J/cm². In some embodiments, the fluence is about 27 J/cm². The skilled person will be able to determine the duration of the exposure required based on the power of the radiation.

Suitable durations may include, but are not limited to, from about 10 seconds to 1 hour (and all integer seconds and minutes therebetween), including about 10 seconds to 30 minutes, about 30 seconds to 20 minutes, 1 minute to 10 minutes or 3 minutes to 7 minutes. In some embodiments, the duration of exposure is about 1, 2, 3, 4, 5, 6, 7, 8, 9, 10, 11, 12, 13, 14 or 15 minutes.

Suitably, the palpebral conjunctiva is exposed to UV-A radiation (320 nm to 400 nm, for example 365 nm) at an irradiance of about 3 to about 6 mW/cm², for example about 3 mW/cm² or about 6 mW/cm².

In some embodiments, the irradiation may be carried out at high irradiance, for example at 30-45 mW/cm² for 1 to 4 minutes, for example 2 to 3 minutes. In some embodiments the radiant exposure, or fluence, is from about 5 to 8 J/cm², for example from about 5.4 to 7.2 J/cm².

The skilled person will appreciate that there will be an upper limit to the amount of irradiation that is considered useful. It is considered that irradiation levels of up to and including 150 mW/cm², for example 150 mW/cm², may be used safely. Irradiation at a level of 150 mW/cm² should be carried out for up to about 4 minutes, or up to about 3 minutes; for example 1 to 4 minutes, 1 to 3 minutes, 2 to 3 minutes, 1 to 2 minutes, or about 1, 2 or 3 minutes.

In some embodiments, the radiation is applied using a beam profile of up to about 15 mm (including about 10, 11, 12, 13, 14 and 15 mm), for example, at a distance of from about 10 mm to about 30 mm (and all integer mm therebetween) from the surface of the palpebral conjunctiva. In some embodiments, the radiation is applied using a beam profile of greater than 12 mm at 10 mm from a surface of the palpebral conjunctiva. A skilled person will appreciate that narrower radiation beams, such as about 11 mm, may be used if the radiation source is repositioned at time intervals to ensure that the entire surface of the palpebral conjunctiva is irradiated.

The delivery of the irradiation may be continuous or pulsed, especially continuous. While the entire surface of the exposed palpebral conjunctiva may be irradiated, in some embodiments, the palpebral conjunctiva is irradiated substantially where the photosensitizer has been applied.

The skilled person will readily appreciate that the wavelength of the photo-activating radiation will depend on the photosensitizer used. In some embodiments, the photo-activating radiation is radiation with a wavelength in the range of from about 300 to about 500 nm (and all integers therebetween) or a wavelength in the range of from about 300 to about 400 nm, particularly UV-A radiation or radiation with a wavelength in the range of from about 320 to about 400 nm. In some embodiments, the radiation has a wavelength in the range of from about 330 to 390, 340 to 380, 350 to 370 or 360 to 370 nm. In particular embodiments, the photo-activating radiation is radiation with a wavelength of about 320, 325, 330, 335, 340, 345, 350, 355, 360, 365, 370, 375, 380, 385, 390, 395 or 400 nm; especially about 355, 360, 365, 370 or 375 nm. In particular embodiments, the photo-activating radiation is radiation with a wavelength of about 365 nm.

In some preferred embodiments, an eye shield or protector, such as a metallic eye protector, is placed under the eyelid and against the anterior aspect of the eye globe prior to irradiation to protect the eye globe from radiation.

In further embodiments, the methods comprise irradiation in the presence of one or more crosslinking agents, preferably a pharmaceutically acceptable crosslinking agent. The skilled person will understand that a crosslinking agent will be preferably substantially non-irritant and non-toxic. A skilled person will be well aware of suitable crosslinking agents. In some embodiments, the crosslinking agent is O₂ gas. For example, in some embodiments, irradiation is carried out in the presence of O₂ gas to provide O₂ concentrations greater than those present in normal atmospheric conditions or in the palpebral conjunctiva. In some embodiments, the additional O₂ is provided at the site of crosslinking during irradiation. In some embodiments O₂ gas is delivered to the surface of the palpebral conjunctiva in the proximity of the irradiation site, for example using a delivery device such as an applicator. In some embodiments, the O₂ gas is humidified prior to use. Methods of generating humidified O₂ gas are known to the skilled person, and include passing the gas flow through a humidifier prior to delivery to the required site.

Any radiation source that is able to deliver radiation of the selected wavelength is suitable for use in the methods of the invention. Radiation sources are known in the art, and suitable sources are commercially available. Radiation sources include those suitable for or intended for corneal crosslinking procedures such as, for example, XLink™ (Optos, Dunfermline, Scotland); Opto XLink™ (Opto Electronica, Sao Carlos, Brazil); CBM Vega XLink Crosslinking System (Carleton Optical, Chesham, UK); LightLink CXL™ (LightMed, San Clemente, CA, USA); UV-XTM 2000 Crosslinking System (IROC Innocross, Zurich, Switzerland); KXL™ CrossLinking System (Avedro Waltham, MA, USA); or an Omnicure UV curing system, such as the OmniCure s1500 UV Curing System (Excelitas Technologies Corp., Waltham, MA, USA). Many of these radiation sources typically have a beam width of approximately 10 to 12 mm. If irradiation of a greater area is necessary, a skilled person will be aware that repositioning of the radiation beam may be required or the radiation source instrument may be modified to produce a greater beam width, such as up to about 15 mm.

In preferred embodiments, the photosensitizer initiates crosslinking of collagen in the tarsal plate tissue in response to photo-activating radiation.

In some embodiments, the method comprises, consists or consists essentially of the steps of:
a) anesthetizing an eyelid of the subject;
b) sterilizing the eyelid;
c) everting the eyelid to expose a palpebral conjunctiva of the eye;
d) applying to at least part of the exposed palpebral conjunctiva a photosensitizer that initiates crosslinking in response to photo-activating radiation;
e) applying directly to the exposed palpebral conjunctiva a radiation beam (e.g. UV-A) to effect crosslinking;
f) rinsing the exposed palpebral conjunctiva to remove residual photosensitizer; and
g) reverting the eyelid to its natural position.

In some embodiments, the method comprises, consists or consists essentially of the steps of:
a) administering a local anesthetic (e.g. oxybuprocaine 0.4% eye drops) to the surface of an eye of the subject to be treated;
b) cleaning the periocular area of the eye, including the upper and lower eyelids, using a solution (e.g. chlorhexidine 0.1% or iodine 0.5%);
c) placing a corneo-scleral shield over the ocular surface;
d) everting the eyelid to expose a palpebral conjunctiva of the eye (e.g. using a lid retractor);
e) applying an opaque surgical drape with a central aperture (e.g. about 10×20 mm in size) over the everted eyelid;
f) applying to the exposed palpebral conjunctiva drops of a photosensitizer that initiates crosslinking in response to photo-activating radiation;
g) applying directly to the exposed palpebral conjunctiva a radiation beam (e.g. UV-A) to effect crosslinking;
h) rinsing the exposed palpebral conjunctiva (e.g. with a saline solution) to remove residual photosensitizer;
i) removing the drape and corneo-scleral shield; and
j) reverting the eyelid to its natural position.

In some embodiments, the method comprises, consists or consists essentially of the steps of:
a) administering a local anesthetic (e.g. oxybuprocaine 0.4% eye drops) to the surface of an eye of the subject to be treated;
b) placing the subject in a supine position;
c) cleaning the periocular area of the eye, including the upper and lower eyelids, using a sterile solution (e.g. chlorhexidine 0.1% or iodine 0.5%);
d) placing a corneo-scleral shield over the ocular surface;
e) everting the eyelid to expose a palpebral conjunctiva of the eye (e.g. using a sterile lid retractor);
f) applying an opaque sterile adhesive surgical drape with a central aperture (e.g. about 10×20 mm in size) over the everted eyelid;
g) applying to the exposed palpebral conjunctiva drops of a sterile photosensitizer solution (e.g. aqueous solution of riboflavin 5′-phosphate monosodium salt) that initiates crosslinking in response to photo-activating radiation to cover the exposed palpebral conjunctiva and leaving the photosensitizer in contact with the exposed palpebral conjunctiva for about 3 to 60 minutes, optionally including applying further drops as needed during the desired time period to keep the exposed palpebral conjunctiva wet and covered with the solution;
h) applying directly to the exposed palpebral conjunctiva a UV-A radiation beam to effect crosslinking, optionally in the presence of O₂ gas delivered close to the irradiation site, for example for 1 to 60 minutes, 3 to 60 minutes, 6 to 60 minutes, 1 to 6 minutes, 1 to 4 minutes, or about 3 minutes depending on the irradiance level;
i) rinsing the treated exposed palpebral conjunctiva (e.g. with a saline solution) to remove residual photosensitizer;
j) removing the drape and corneo-scleral shield; and
k) reverting the eyelid to its natural position.

In some embodiments, the method is performed as follows. A local anesthetic such as oxybuprocaine (0.4%) eyedrops is administered to the ocular surface to achieve effective anesthesia. The subject is then placed a supine position, for example, on a flat operating table with appropriate head support to maintain stability and proper alignment during the procedure. The treatment periocular area, including the upper and lower eyelids, is then cleaned using a sterile solution of, for example, chlorhexidine 0.1% or iodine 0.5%, to minimize the risk of infection and ensure a sterile field. A large, sterile surgical corneo-scleral shield may then be placed over the ocular surface to protect the cornea, conjunctiva and sclera from potential radiation exposure. The treatment eyelid (upper or lower) is everted using a sterile lid retractor to expose the palpebral conjunctiva, facilitating effective application of treatment. A sterile adhesive surgical drape with a central aperture measuring 10×20 mm is applied over the everted eyelid. The drape is preferably opaque to prevent the penetration of UV radiation to non-targeted adjacent tissues and ensure precise treatment localization. A sterile photosensitizer solution (e.g. 1 to 5 ml of aqueous solution of riboflavin 5′-phosphate monosodium salt, 0.1 to 0.5% w/w) is applied using a sterile dropper to the treatment area where it remains for from 3 to 60 minutes, whilst keeping the treatment area consistently wet and covered with the solution during this time. Optionally, excess solution is removed using a sponge device, e.g. a Week-Cell™ cellulose eye spear sponge. Alternatively, a neurosurgical patty (25×12 mm) trimmed to an appropriate size, is soaked for about 5 minutes in the aqueous solution of the photosensitizer, and then placed onto the palpebral conjunctiva, where it remains for from 3 to 60 minutes prior to removal. A beam of UV-A radiation is then delivered to the treatment area for a duration depending on the irradiance level. The UV-A radiation may be provided by a commercially available UV corneal crosslinking machine as described elsewhere herein. The duration of the irradiation will depend on the wavelength and power of the radiation beam and can be readily determined by the skilled person. Typical irradiation times at an irradiance of 3 mW/cm² are about 3 to 60 minutes, suitably from about 10 to 50 minutes, about 15 to 45 minutes, about 20 to 40 minutes, about 30 to 40 minutes, about 20 to 30 minutes or about 30 minutes. Typical irradiation times at an irradiance of 6 mW/cm² are about 3 to 60 minutes, suitably from about 10 to 40 minutes, about 15 to 45 minutes, about 20 to 40 minutes, about 20 to 30 minutes, about 15 to 20 minutes or about 30 minutes. Humidified O₂ gas can be delivered at the site of crosslinking if desired. After irradiation, the treatment area is gently rinsed with balanced saline solution to remove residual photosensitizer. The drape and corneo-scleral shield are then removed and the eyelid is reverted to its natural position.

If required, the methods may be repeated. For example, the methods may be performed one or more times, such as two times, three times or four times over a time period of, for example, 3 months, 6 months, one year, two years, three years and the like.

Additional materials (e.g. surgical materials) are well known to the skilled person and are readily available from commercial sources. Suitable local anesthetics for anesthetizing the eyelid are well known to the skilled person and include, for example, Alcaine™ (proparacaine hydrochloride), Naropin™ (ropivicane hydrochloride), Marcaine™ (bupivacaine hydrochloride) and Novesin™ (benoxinate, oxybuprocaine hydrochloride). Suitable antiseptics include Betadine™ (povidone/iodine) and Betasept™ (chlorhexidine).

In some embodiments, the eyelid laxity is mild or moderate eyelid laxity.

Also provided is a use of a photosensitizer that initiates crosslinking in response to photo-activating radiation in the manufacture of a medicament for treating or inhibiting the development or progression of eyelid laxity in a subject, wherein the photosensitizer is to be applied to at least part of an exposed palpebral conjunctiva of an eye of the subject, and the exposed palpebral conjunctiva is to be irradiated with photo-activating radiation to initiate crosslinking in tarsal plate tissue underlying the palpebral conjunctiva.

Suitable embodiments of the photosensitizer, radiation and exposure of the palpebral conjunctiva are as described supra. In some embodiments, the use further comprises exposing a palpebral conjunctiva of an eye of the subject prior to application of the photosensitizer.

In another aspect, there is provided a use of a photosensitizer that initiates crosslinking in response to photo-activating radiation for treating or inhibiting the development or progression of eyelid laxity in a subject, wherein the photosensitizer is to be applied to at least part of an exposed palpebral conjunctiva of an eye of the subject, and the exposed palpebral conjunctiva is to be irradiated with photo-activating radiation to initiate crosslinking in tarsal plate tissue underlying the palpebral conjunctiva.

Suitable embodiments of the photosensitizer, radiation and exposure of the palpebral conjunctiva are as described supra. In some embodiments, the use further comprises exposing a palpebral conjunctiva of an eye of the subject prior to application of the photosensitizer.

In a further aspect, there is provided a photosensitizer that initiates crosslinking in response to photo-activating radiation for use in treating or inhibiting the development or progression of eyelid laxity in a subject, wherein the photosensitizer is applied to at least part of an exposed palpebral conjunctiva of an eye of the subject, and the exposed palpebral conjunctiva is irradiated with photo-activating radiation to initiate crosslinking in tarsal plate tissue underlying the palpebral conjunctiva.

Suitable embodiments of the photosensitizer, radiation and exposure of the palpebral conjunctiva are as described supra. In some embodiments, the use further comprises exposing a palpebral conjunctiva of an eye of the subject prior to application of the photosensitizer.

Also provided is a method of crosslinking tarsal plate tissue in an eye comprising, consisting or consisting essentially of:
a) exposing a palpebral conjunctiva of an eye;
b) applying to at least part of the exposed palpebral conjunctiva a photosensitizer that initiates crosslinking in response to photo-activating radiation; and
c) irradiating the exposed palpebral conjunctiva with photo-activating radiation to initiate crosslinking in tarsal plate tissue underlying the palpebral conjunctiva.

Suitable embodiments of the photosensitizer, radiation and exposure of the palpebral conjunctiva are as described supra. In particular embodiments, the photosensitizer is topically applied.

Any one of the methods and uses described herein may involve the application of an effective amount of the photosensitizer and photo-activating radiation, or the use of a system or kit as described elsewhere herein. In some embodiments, any one of the methods and uses are non-invasive and do not involve surgery.

In order that the invention may be readily understood and put into practical effect, particular preferred embodiments will now be described by way of the following non-limiting examples.

### EXAMPLES

### Example 1-Ex Vivo Crosslinking of Human Tissue

### Materials and Methods

### Tarsoconjunctival Tissue Harvesting Technique

The tarsoconjunctival tissue was harvested from the upper eyelids (right and left) of fresh human cadavers, that is, never frozen, but only refrigerated (at ˜4° C.) prior to necropsy, for no longer than 72 hours. The harvesting took place within 1-2 hours, at which stage the body temperature did not reach over 10-12° C.

The eyelid was everted on a Desmarres retractor using a 4.0 nylon stay suture. A horizontal incision was made along the entire length of the tarsal plate and overlying conjunctiva, 2 mm above the lid margin, using a No. 15 blade. Vertical incisions were placed at the medial and lateral borders. The tarsoconjunctival tissue was gently elevated and dissected from the orbicularis muscle, the levator aponeurosis, and orbital septum attachments with Westcott scissors. The conjunctiva was cut at the superior edge of the tarsal plate. Care was taken to avoid crush artifacts of the harvested tarsoconjunctival tissue specimens.

### Preparation of the Tarsal Tissue

The samples were placed in normal saline (0.9%) and transported immediately to the laboratory to make sure that they would be prepared within 24 hours of harvest for irradiation and biomechanical evaluation. Using an optical microscope, the tarsal plate was cleaned of any residual muscle tissue. The strongly adherent palpebral conjunctiva was preserved.

### Irradiation Procedure

The 365 nm ultraviolet radiation was generated by the ultraviolet Curing System OmniCure 1500 (Excelitas Technologies Corp., Waltham, MA, USA), while a radiometer Dymax ACCU-CAL 50 (Dymax Corp., Torrington, CT, USA) was used to monitor the irradiance at the exposure site on samples. The required irradiance level was assured by adjusting the distance between the source and the sample's surface.

Each specimen was placed on a cushion of hyaluronate gel (Ophteis, Bio-Tech AB, Kungsbacka, Sweden), with the tarsal plate facing down and leaving only the conjunctival side exposed for treatment. About 3 or 4 drops of 0.1% riboflavin 5′-monophosphate sodium salt in phosphate-buffered saline were applied to the conjunctival surface and left in place for 5 minutes. Each specimen was then exposed to UV-A radiation (365 nm) for 3 minutes at an irradiance of 75 mW/cm².

### Mechanical Testing

The tarsoconjunctival samples were mechanically assessed in the BioTester 5000 (CellScale Co., Waterloo, ON, Canada). This instrument allows accurate evaluation of the mechanical properties of a variety of biological samples. It is equipped with 4 actuators that allow biaxial measurements, load cells of various capacities, systems of rakes for specimen mounting, and user interface software. The 23-N load cell was used during the entire evaluation. Due to the shape of the tarsal specimens, measurements were required to be conducted in the uniaxial mode, using only 2 of the actuators. The dimensions of tested specimens were measured with a digital caliper of accuracy 0.01 mm (model PRECISE PS7215, Burg-Wachter K G, Watter, Germany), prior to each measurement and are shown in Table 1. The specimens were stored in phosphate-buffered saline in a refrigerator and tested within 24-48 hours of harvesting.

For testing, the specimens were clamped along one longitudinal axis between 2 opposite arms, with a working distance of 7 mm, and submerged into the instrument's bath filled with phosphate-buffered saline and maintained at 37±1° C. for the duration of the measurement. Tensile preloading was applied for 60 seconds to achieve a force of 100 mN that was maintained for 5 minutes. After this preconditioning, 15 repeated cyclic tensile loading/unloading tests were performed at 10% displacement (strain) at a rate of 0.33%/s, and then back to zero. The last cycle (the 15th) was used to calculate Young's modulus (YM) and the stress for each specimen. The specimens were then removed from the BioTester, exposed to irradiation, and reevaluated in the BioTester after 1-hour rest in phosphate-buffered saline. Samples no. 1 and no. 2 were used to check the level of reproducibility of measuring in the BioTester, and they were not further exposed to irradiation.

The CellScale Labjoy 2.0 software was used to compute numerical values for YM and stress at a 10% displacement (strain). This level of strain was chosen to ensure that calculations were made within the linear region of the stress-strain plot.

### Histology

The histopathological analysis was conducted on 2 upper eyelid tarsal plates, each harvested from a different subject. Each plate was sectioned into 2 specimens; one specimen was irradiated according to the above protocol, while the other was left intact and served as the control. These 4 samples, allocated for histology only, were not subjected to biomechanical testing and, therefore, are not included in Table 1. To avoid confusion the following code numbers were assigned to the histology samples (continuing those in Table 1): no. 16 (source: male subject, 86 years, not irradiated), no. 17 (the same origin as no. 16, irradiated), no. 18 (source: female subject, 55 years, not irradiated), and no. 19 (the same origin as no. 18, irradiated). In other words, the pairs of histologic samples (i.e., 16/17 and 18/19) are each from a different cadaver, and they were not subjected to mechanical testing.

The excised specimens were stored in formaldehyde solution of 4%, prepared from 10% Neutral Buffered Formalin (Epredia, Breda, the Netherlands). Both the irradiated and nonirradiated tissue samples were placed in histological cassettes with holes (˜6×1 mm) (Bio-Optica, Milano, Italy) to be sectioned in the sagittal plane. The contained specimens were dehydrated in a series of graded ethanol solutions (70°, 96° and absolute), and then embedded in paraffin (melting point 56° C.). The paraffin blocks were cut into 2 μm sections using a semiautomated microtome Model HM340E (Thermo Fisher Scientific, Waltham, MA, USA) with MX35 ULTRA blades (Kai Industries, Gifu, Japan). The sections were then mounted on histological slides (Superfrost, Epredia, Breda, the Netherlands), deparaffined with xylene, and hydrated with decreasing concentrations of ethanol.

Sections from each sample were stained individually with 3 different staining techniques, hematoxylin & eosin, Masson trichrome, and Van Gieson stain. An automated stainer (Gemini AS, Thermo Fisher Scientific, Waltham, MA, USA) was used for hematoxylin & eosin staining, while the other 2 procedures were manual. The Masson trichrome kit (MDSS GmbH, Hannover, Germany) was used following deparaffination; then hematoxylin was applied for 5 minutes, rinsed with running water for 5 minutes, treated successively with acid fuchsin and phosphomolybdic acid, rinsed with water, treated with aniline blue, followed by ethanolic dehydration, clarification with xylene, and mounting on glass slides. Van Gieson kit (MDSS GmbH, Hannover, Germany) was used for staining, and after deparaffination, hematoxylin and picrofuchsin were applied successively, the sections were dehydrated, clarified with xylene, and mounted.

The slides were examined by 2 experienced pathologists with a Zeiss Axio Lab A1 light microscope (Carl Zeiss Microscopy GmbH, Jena, Germany), and photocaptured using the Zeiss ZenPro 3.2 software (Carl Zeiss Microscopy GmbH, Jena, Germany).

### Data Processing

The GraphPad Prism (v. 6.0) software was employed for statistical comparison of the final values of stress and YM, before and after irradiation, applying the Wilcoxon matched pair rank test for n=13.

### Results

A total of 17 upper eyelid tarsoconjunctival tissue specimens were harvested from 12 cadavers. Thirteen samples were subjected to mechanical evaluation before and after irradiation. The remaining 4 samples were not irradiated, as 2 of them were used to assess the measurements reproducibility in the BioTester, while the other 2 were used for histopathological analysis.

Table 1 presents data related to the origin and size of the tarsal issue specimens, and their measured biomechanical properties (YM, stress) reflecting the stiffness and, respectively, the strength, before and after irradiation according to the protocol described above.

The values measured in the BioTester 5000 for YM and stress, calculated within the linear region of the stress-strain plot at a displacement of 10%, indicate unequivocally the existence of UV-A radiation-induced stiffening and strengthening effects on tarsal tissue.

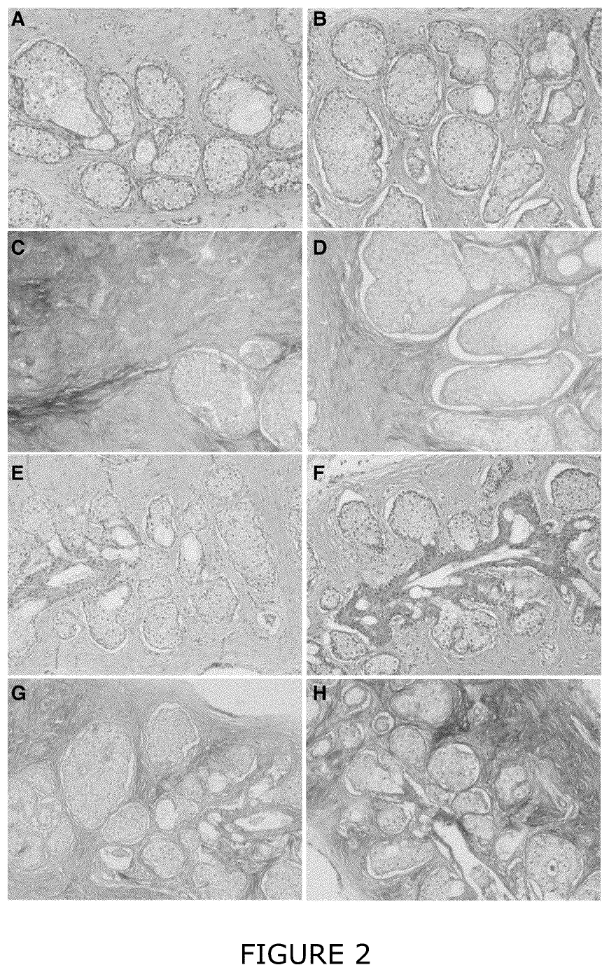

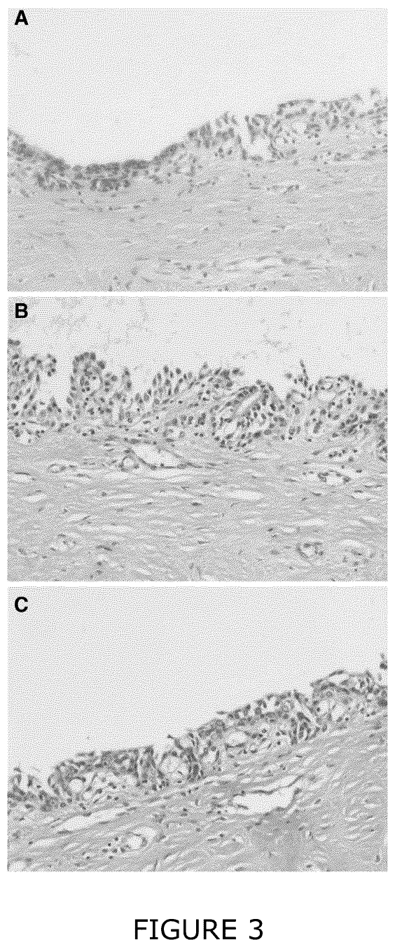

Histology revealed the absence of any tissue damage caused by irradiation. Hematoxylin & eosin staining showed dense, fibrous connective tissue surrounding normal Meibomian gland acini, and occasional inflammatory cells both in controls (FIGS. 2A and E) and in irradiated tarsal plates (FIGS. 2B and F). Van Gieson staining showed normal collagen network (red) and Meibomian glands (yellow) both in nonirradiated (FIG. 2C) and in irradiated tarsal plates (FIG. 2D). Finally, Masson trichrome staining demonstrated normal connective tissue (blue), meibocytes' nuclei (brown), and cytoplasm of cells lining the excretory ducts (red-brown) both in nonirradiated (FIG. 2G) and in irradiated tarsal plates (FIG. 2H). Hematoxylin & eosin staining was employed to examine the palpebral conjunctiva before and after irradiation (FIG. 3). When comparing the images of a nonirradiated sample (FIG. 3A) with those of 2 different specimens that were irradiated (FIGS. 3B and C), the nonkeratinized stratified squamous conjunctival epithelium appears normal. The surface epithelial cells and mucocytes displayed a normal aspect retaining their polarity, polygonality, eosinophilic cytoplasm, and preserved nuclei. While in some samples the conjunctival epithelium appeared partially detached due to prior surgical handling, it was concluded that the exposure of palpebral conjunctiva to UV-A radiation did not result in its damage.

TABLE 1
DESCRIPTION OF UPPER EYELID TARSAL SPECIMENS AND EFFECT
OF IRRADIATION ON THEIR MECHANICAL PROPERTIES

| | | | Native tarsus | | | | |
|---|---|---|---|---|---|---|---|
| Sample | | Age | Dimensions (mm) | | | Young's | Stress |
| no. | Gender | (at death) | Length | Width | Thickness | modulus (kPa)* | (kPa)* |
| 1† | F | 66 | 25.24 | 9.37 | 1.38 | 7.54 | 26.45 |
| 1†‡ | | | | | | 9.11 | 24.21 |
| 2† | F | 69 | 20.92 | 7.33 | 1.41 | 6.00 | 12.00 |
| 2†‡ | | | | | | 6.84 | 19.64 |
| 3§ | F | 38 | 26.05 | 8.34 | 1.37 | 8.18 | 19.69 |
| 4¶ | F | 66 | 22.79 | 7.91 | 1.32 | 8.20 | 25.95 |
| 5¶ | F | 69 | 22.12 | 8.69 | 1.50 | 5.72 | 16.19 |
| 6 | F | 76 | 25.64 | 8.86 | 1.40 | 7.68 | 24.43 |
| 7 | | | 23.81 | 8.41 | 1.45 | 4.96 | 15.66 |
| 8 | M | 54 | 26.09 | 9.60 | 1.30 | 11.23 | 36.06 |
| 9 | | | 26.10 | 8.90 | 1.10 | 14.77 | 47.60 |
| 10 | M | 56 | 27.10 | 9.64 | 1.40 | 11.78 | 38.46 |
| 11 | | | 28.38 | 8.72 | 1.55 | 7.21 | 27.74 |
| 12 | M | 67 | 26.06 | 8.99 | 1.47 | 7.16 | 20.73 |
| 13 | | | 24.31 | 9.16 | 1.34 | 10.04 | 30.63 |
| 14 | M | 86 | 24.59 | 8.98 | 1.21 | 6.80 | 23.10 |
| 15 | | | 27.10 | 8.75 | 1.39 | 3.49 | 11.92 |
| Average | | 64 | 25.40 | 8.84 | 1.37 | 8.25∥ | 26.01∥ |
| SD | | 15 | 1.79 | 0.48 | 0.12 | 3.05 | 10.11 |

| | Irradiated tarsus | | | | | |
|---|---|---|---|---|---|---|
| Sample | Dimensions (mm) | | | Young's | Stress | % Increase |
| no. | Length | Width | Thickness | modulus (kPa)* | (kPa)* | in YM |
| 1† | — | — | — | — | — | — |
| 1†‡ | — | — | — | — | — | — |
| 2† | — | — | — | — | — | — |
| 2†‡ | — | — | — | — | — | — |
| 3§ | 25.90 | 8.30 | 1.31 | 25.08 | 80.69 | 207 |
| 4¶ | 22.79 | 7.91 | 1.32 | 30.10 | 94.91 | 267 |
| 5¶ | 22.12 | 8.69 | 1.50 | 19.55 | 58.84 | 242 |
| 6 | 25.49 | 8.32 | 1.41 | 24.01 | 85.70 | 212 |
| 7 | 23.27 | 7.98 | 1.27 | 24.66 | 88.81 | 397 |
| 8 | 25.50 | 9.41 | 1.30 | 27.71 | 98.32 | 147 |
| 9 | 26.91 | 9.02 | 1.36 | 37.40 | 141.57 | 153 |
| 10 | 27.81 | 9.19 | 1.23 | 21.15 | 76.91 | 80 |
| 11 | 27.71 | 9.07 | 1.43 | 25.50 | 89.30 | 254 |
| 12 | 25.57 | 9.03 | 1.28 | 22.70 | 76.80 | 217 |
| 13 | 25.90 | 9.37 | 1.19 | 17.81 | 55.07 | 77 |
| 14 | 26.90 | 7.81 | 0.87 | 25.32 | 88.44 | 273 |
| 15 | 27.04 | 8.36 | 0.97 | 20.06 | 64.05 | 475 |
| Average | 25.61 | 8.65 | 1.26 | 24.70∥ | 84.57∥ | 199 |
| SD | 1.84 | 0.57 | 0.18 | 5.11 | 21.77 | 67 |

*Calculated at 10% displacement (strain).
†This sample was not irradiated.
‡Second measurement (after 1-hour rest).
§The measurements for the [symbol] could not be validated due to incidental failure of the instrument
¶The [symbol] samples for no. 4 and no. 5 are, respectively, samples no. 1 and no. 2.
∥Statistically significant difference: p < 0.0002 (n = 13). [symbol]
indicates data missing or illegible when filed

### Example 2-Ex Vivo Thermographic Analysis of the Eyelid Conjunctival Surface Exposed to UV-A Radiation During Crosslinking of Tarsal Collagen

### Materials and Methods

Eyelids were harvested on site postmortem from sheep cadavers supplied by Brisbane Valley Meats Pty Ltd, Esk, Queensland, Australia, which is an abattoir unit operating under the rules of the Australian Code of Practice of the Animal Welfare Standards for Livestock Processing Establishments (2001) and the Australian Animal Welfare Standards and Guidelines for Sheep (2016).

Riboflavin 5′-phosphate monosodium salt, supplied by Cayman Chemical Co. (Ann Arbor, Michigan, USA), was dissolved in phosphate-buffered saline (PBS) to a concentration of 0.25% w/v, and the solution was applied to the tissue specimens in order to duplicate the conditions during an actual photocrosslinking procedure.

KIMTECH Sciences (Blacktown, New South Wales, Australia) supplied the KimwipeR blotting paper tissues.

Twelve full-thickness eyelid specimens were excised postmortem from the ocular adnexa of six pairs of sheep eyes that were refrigerated for no longer than 24 h. For convenience, the eyelashes were trimmed. The specimens were soaked for 30 min at room temperature in a solution of riboflavin 5′-phosphate monosodium salt in PBS (0.25% w/v). After removal from the solution, each sample was placed on a Petri dish with the conjunctival side facing upwards, and the excess of liquid was gently blotted off with KimwipeR paper tissues.

A thermal camera model FLIR C5 (FLIR Systems Australia Pty Ltd, Mulgrave, Victoria, Australia) was used for the thermographic analysis, with a thermal sensitivity of <0.07° C. As the source of radiation, the UV Curing System OmniCureR 1500 (Excelitas Technologies Corp., Waltham, MA, USA) was employed. To determine the level of irradiance at the exposure site, a radiometer Dymax ACCU-CAL 50 (Dymax Corp., Torrington, CT, USA) was used for the pre-irradiation adjustment of the distance between the UV source and tissue surface. The samples were each exposed for 3 min to UV-A radiation with a wavelength of 365 nm, at the irradiances of 45, 75, 150, or 250 mW/cm², corresponding respectively to fluences of 8.1, 13.5, 27, or 45 J/cm². Three eyelid specimens were used for each irradiance, and the data points were plotted as mean values±s.d.

The infrared camera was mounted on a tripod and placed at a distance of ˜20 cm from the sample. The measurement spot (the ‘spotmeter’) on the camera screen was positioned onto the surface of the tissue as centrally as possible. During each round of irradiation, the images containing information regarding the temperature at the target spot were recorded every 30 s, also including the baseline measurements prior to each irradiation episode.

### Results

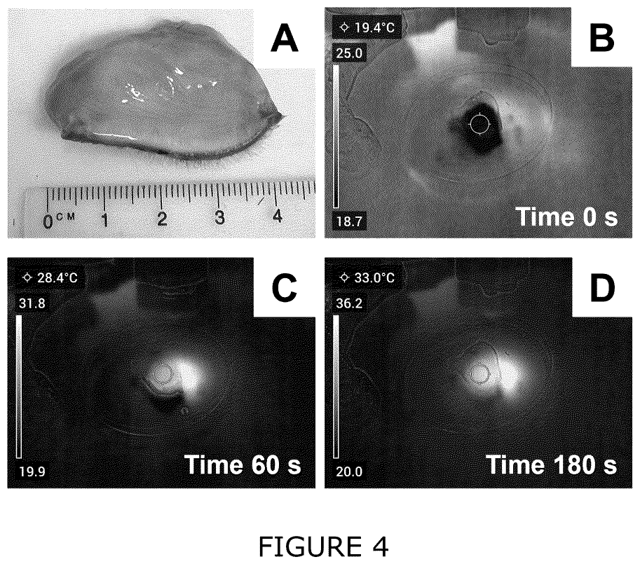

FIG. 4A shows a photograph of a sheep eyelid tissue sample that was soaked for 30 min in the photosensitizer solution, ready to be exposed to UV-A radiation. FIG. 4B displays the thermographic image of a tissue specimen recorded before commencing the irradiation procedure, while FIGS. 4C and 4D comprise typical thermographic images recorded at the end of an exposure protocol, exemplified for an irradiance of 250 mW/cm² at two different exposure times.

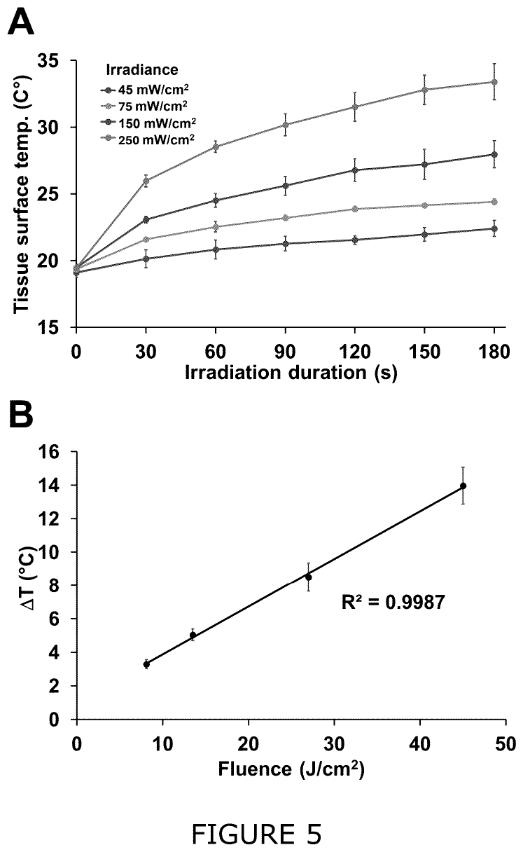

As expected, the temperatures recorded on the surface of tarsal conjunctiva increased both with the exposure time and with the irradiance (FIG. 5A). Furthermore, there was a linear dependence between fluence and the temperature increase (AT) (FIG. 5B).

The baseline temperatures (prior to irradiation) were very similar in all experiments, i.e. between 18.7 and 19.7° C., or 19.3+0.3° C.

The exposure to UV-A radiation (with a wavelength of 365 nm) led to increased temperature on the surface of tarsal conjunctiva, as shown in the eyelid of an ovine model. The post-exposure temperature increments were low, and there was an ideal linear dependence between them and the fluence of the incident radiation. Thermal effects of this magnitude are not evocative of tissue damage occurring for the ranges of irradiance (intensity) and fluence (radiant exposure) applicable in the treatment of eyelid laxity.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

The claims defining the invention are as follows:

1. A method of treating or inhibiting the development or progression of eyelid laxity in a subject comprising:
a) exposing a palpebral conjunctiva of an eye;
b) applying to at least part of the exposed palpebral conjunctiva a photosensitizer that initiates crosslinking in response to photo-activating radiation; and
c) irradiating the exposed palpebral conjunctiva with photo-activating radiation to initiate crosslinking in tarsal plate tissue underlying the palpebral conjunctiva.

2. The method according to claim 1, wherein the photosensitizer is in solubilized form.

3. The method according to claim 1, wherein the photosensitizer is selected from the group consisting of riboflavin, acridine orange, Quantacure QTX, protoporphyrin IX and pharmaceutically acceptable salts, derivatives and solvates thereof.

4. The method according to claim 1, wherein the photosensitizer absorbs radiation at a wavelength in the range of from about 300 to about 500 nm.

5. The method according to claim 1, wherein the photosensitizer is riboflavin or a pharmaceutically acceptable salt, derivative or solvate thereof.

6. The method according to claim 1, wherein the photosensitizer is riboflavin 5′-phosphate or a pharmaceutically acceptable salt or solvate thereof.

7. The method according to claim 1, wherein the photo-activating radiation is radiation with a wavelength in the range of from about 300 to about 500 nm.

8. The method according to claim 7, wherein the photo-activating radiation is UV-A radiation.

9. The method according to claim 7, wherein the photo-activating radiation is radiation with a wavelength of about 365 nm.

10. The method according to claim 1, wherein the palpebral conjunctiva is exposed by everting an upper or lower eyelid of the subject to expose the palpebral conjunctiva.

11. The method according to claim 10, wherein the upper or lower eyelid of the subject is everted using a lid retractor.

12. The method according to claim 1, wherein the method further includes delivery of O₂ gas at a site of irradiation.

13. The method according to claim 1, wherein the photosensitizer is riboflavin 5′-phosphate or a pharmaceutically acceptable salt or solvate thereof, the photo-activating radiation is radiation with a wavelength in the range of from about 300 to about 500 nm and the palpebral conjunctiva is exposed by everting an upper or lower eyelid of the subject to expose the palpebral conjunctiva.

14. The method according to claim 13, wherein the photo-activating radiation is radiation with a wavelength of about 365 nm.

15. Use of a photosensitizer that initiates crosslinking in response to photo-activating radiation for treating or inhibiting the development or progression of eyelid laxity in a subject, wherein the photosensitizer is to be applied to at least part of an exposed palpebral conjunctiva of an eye of the subject, and the exposed palpebral conjunctiva is to be irradiated with photo-activating radiation to initiate crosslinking in tarsal plate tissue underlying the palpebral conjunctiva.
